(19) United States
(12) Patent Application Publication
(10) Pub. No.: US 2024/0175067 A1
(43) Pub. Date: May 30, 2024

(54) A METHOD OF DIAGNOSING DEPRESSION IN A SUBJECT

(71) Applicant: University of Ulster, Coleraine, County Londonderry (GB)

(72) Inventors: Benjamin WINGFIELD, Coleraine, County Londonderry (GB); Tony BJOURSON, Coleraine, County Londonderry (GB); Sonya COLEMAN, Coleraine, County Londonderry (GB); Martin MCGINNITY, Coleraine, County Londonderry (GB); Elaine MURRAY, Coleraine, County Londonderry (GB); Coral LAPSLEY, Coleraine, County Londonderry (GB); Andrew MCDOWELL, Coleraine, County Londonderry (GB); Siobhan O'NEILL, Coleraine, County Londonderry (GB); Margaret MCLAFFERTY, Coleraine, County Londonderry (GB)

(21) Appl. No.: 18/009,960

(22) PCT Filed: Jun. 14, 2021
(86) PCT No.: PCT/EP2021/065987

§ 371 Date: Dec. 12, 2022

(30) Foreign Application Priority Data
Jun. 12, 2020 (GB) 2009010.6

(51) Int. Cl. C12Q 1/06

(52) U.S. Cl. CPC C12Q 1/06

(57) ABSTRACT

The present invention relates to a method of diagnosing depression in a subject. Specifically, the present invention relates to diagnosing depression in a subject by providing a sample of the microbiome from the subject; determining the quantitative level or presence of one or more bacterial species in the sample; and diagnosing depression based on the quantitative level or presence of the one or more bacterial species in the sample.

### BACKGROUND TO THE INVENTION

Depression is a highly-prevalent, complex mental health disorder characterised by a range of debilitating symptoms. Depression is diagnosed by General Practitioners, using measures in line with the Diagnostic and Statistical Manual of Mental Disorders (DSM) criteria, along with the integration of individual patient information and background, ultimately reliant on patient self-reporting and clinical judgement. No empirical diagnostic tests are currently in clinical use and, despite ongoing research; the biological basis of depression is poorly understood, possibly due to the heterogeneous pathophysiology.

A number of theories of depression have been proposed including neurotransmitter deficiencies, changes to neurotrophic levels, structural brain abnormalities, immune system dysregulation, and circadian rhythm disruption. Despite a number of hypotheses, no single biological mechanism or environmental factor is conclusive.

The role of the microbiota inhabiting the human gastrointestinal tract in the regulation of the central nervous system—a complex network known as the gut-brain axis—has been predominantly investigated in preclinical studies. While these models have provided valuable brain-gut pathway level information, it is still unknown if animal models of microbial dysregulation can capture the complexity of human brain disorders such as depression. Clinical studies are only emerging, and almost exclusively studied in gut microbiota. Along with the gut, the oral microbiome is also one of the most diverse microbiomes in the human body and similarly plays an important role in health and disease. The mouth is highly vascularized and bacteraemia due to bacterial translocation across the epithelial mucosa is an everyday event.

However, the collection of faecal samples for such clinical studies comes with a number of challenges for modern large scale-epidemiological studies: Samples cannot be collected on demand and typically need to be collected in the home and transported to the lab; transport and processing must be stringent and standardised as temperature and time to processing effects microbial growth; and additional recruitment barriers exist as potential participants may have objections such as embarrassment or hygiene concerns.

### SUMMARY OF THE INVENTION

According to a first aspect of the present invention, there is provided a method of diagnosing depression in a subject, the method comprising the steps of:
(a) providing a sample of the microbiome from the subject;
(b) determining the quantitative level or presence of one or more bacterial species in the sample; and
(c) diagnosing depression based on the quantitative level or presence of the one or more bacterial species in the sample;

wherein the one or more bacterial species is selected from one or more of the phyla:
(i) Firmicutes;
(ii) Proteobacteria; and
(iii) Bacteroidetes.

Optionally or additionally, the one or more bacterial species is selected from one or more of the phyla:
(iv) Fusobacteria; and
(v) Actinobacteria.

Preferably, the one or more bacterial species is selected from one or more of the phyla:
(i) Firmicutes;
(ii) Proteobacteria;
(iii) Bacteroidetes;
(iv) Fusobacteria; and
(v) Actinobacteria.

Optionally, the one or more bacterial species is selected from the phylum Spirochaetes.

Further optionally, the one or more bacterial species comprise members of the phyla:
(i) Firmicutes;
(ii) Proteobacteria;
(iii) Bacteroidetes;
(iv) Fusobacteria;
(v) Actinobacteria; and
(vi) Spirochaetes.

Optionally, the or each bacterial species is selected from the phylum Firmicutes and is selected from one or more of the genera:
(i) Solobacterium;
(ii) Veillonella;
(iii) Streptococcus;
(iv) Lachnoanaerobaculum;
(v) Selenomonas_3;
(vi) Oribacterium;
(vii) Gemella;
(viii) Stomatobaculum;
(ix) Megasphaera;
(x) Selenomonas;
(xi) Carnobacteria;
(xii) Erysipelotrichia;
(xiii) Mogibacterium; and
(xiv) Granulicatella.

Optionally, the or each bacterial species is selected from the phylum Firmicutes and is selected from the genus Solobacterium.

Preferably, the or each bacterial species is selected from the phylum Firmicutes and is selected from one or more of the genera:
(i) Veillonella;
(ii) Streptococcus;
(iii) Lachnoanaerobaculum;
(iv) Selenomonas_3;
(v) Oribacterium;
(vi) Gemella;
(vii) Stomatobaculum;
(viii) Megasphaera;
(ix) Selenomonas; and
(x) Mogibacterium.

Alternatively, the or each bacterial species is selected from the phylum Firmicutes and is selected from one or more of the genera:
(i) Streptococcus;
(ii) Veillonella;
(iii) Granulicatella;
(iv) Megasphaera;
(v) Gemella;
(vi) Selenomonas_3;
(vii) Gemella; and
(viii) Selenomonas.

Further alternatively, the or each bacterial species is selected from the phylum Firmicutes and is selected from one or more of the genera:
(i) Carnobacteria;
(ii) Erysipelotrichia;
(iii) Streptococcus; and
(iv) Veillonella.

Optionally, the or each bacterial species is selected from the phylum Proteobacteria and is selected from one or more of the genera:
(i) Haemophilus;
(ii) Pasteurella;
(iii) Neisseria;
(iv) Aggregatibacter;
(v) Neisseria ASV 1;
(vi) Neisseria ASV 2; and
(vii) Neisseria ASV 3.

Preferbaly, the or each bacterial species is selected from the phylum Proteobacteria and is selected from one or more of the genera:
(i) Haemophilus; and
(ii) Neisseria.

Optionally, the or each bacterial species is selected from the phylum Proteobacteria and is selected from one or more of the genera:
(i) Haemophilus;
(ii) Neisseria; and
(iii) Aggregatibacter.

Alternatively, the or each bacterial species is selected from the phylum Proteobacteria and is selected from one or more of the genera:
(i) Pasteurella; and
(ii) Neisseria.

Optionally, the or each bacterial species is selected from the phylum Bacteroidetes and is selected from one or more of the genera:
(i) Prevotella;
(ii) Prevotella_7;
(iii) Bergeyella;
(iv) Porphyromonas;
(v) Flavobacteria;
(vi) Alloprevotella; and
(vii) Prevotella_6.

Optionally, the or each bacterial species is selected from the phylum Bacteroidetes and is selected from one or more of the genera:
(i) Prevotella;
(ii) Bergeyella;
(iii) Porphyromonas; and
(iv) Alloprevotella.

Preferably, the or each bacterial species is selected from the phylum Bacteroidetes and is selected from one or more of the genera:
(i) Prevotella; and
(ii) Bergeyella.

Alternatively, the or each bacterial species is selected from the phylum Bacteroidetes and is selected from one or more of the genera:
(i) Prevotella;
(ii) Prevotella_6;
(iii) Porphyromonas; and
(iv) Prevotella_7.

Further alternatively, the or each bacterial species is selected from the phylum Bacteroidetes and is selected from one or more of the genera:
(i) Flavobacteria;
(ii) Porphyromonas; and
(iii) Prevotella.

Optionally, the or each bacterial species is selected from the phylum Fusobacteria and is selected from one or more of the genera:
(i) Leptotrichia; and
(ii) Fusobacterium.

Preferably, the or each bacterial species is selected from the phylum Bacteroidetes and is selected from the genus Fusobacterium.

Optionally, the or each bacterial species is selected from the phylum Actinobacteria and is selected from one or more of the genera:
(i) Rothia;
(ii) Actinomyces; and
(iii) Schaalia.

Preferably, the or each bacterial species is selected from the phylum Actinobacteria and is selected from the genus Rothia.

Optionally, the or each bacterial species is selected from the phylum Spirochaetes and is selected from the genus Treponema_2.

Optionally, the or each bacterial species is selected from the genus Veillonella and is selected from one or more of the species:
(i) Veillonella dispar;
(ii) Veillonella parvula;
(iii) Veillonella rogosae; and
(iv) Veillonella atypica.

Optionally, the or each bacterial species is selected from the genus Streptococcus and is selected from one ore more of the species:
(i) Streptococcus australis;
(ii) Streptococcus infantis;
(iii) Streptococcus sanguinis;
(iv) Streptococcus parasanguinis; and
(v) Streptococcus oralis.

Optionally, the or each bacterial species is selected from the genus Gemella and is selected from one or more of the species:
(i) Gemella haemolysans; and
(ii) Gemella sanguinis.

Optionally, the or each bacterial species is selected from the genus Megasphaera and is selected from the species Megasphaera micronuciformis.

Optionally, the or each bacterial species is selected from the genus Selenomonas and is selected from the species Selenomonas sputigena.

Optionally, the or each bacterial species is selected from the genus Haemophilus and is selected from the species Haemophilus parainfluenzae.

Optionally, the or each bacterial species is selected from the genus Neisseria and is selected from one or more of the species:
(i) Neisseria mucosa;
(ii) Neisseria pharynges;
(iii) Neisseria perflava; and
(iv) Neisseria subflava.

Optionally, the or each bacterial species is selected from the genus Alloprevotella and is selected from the species Alloprevotella tannerae.

Preferably, the or each bacterial species is selected from the genus Alloprevotella and is selected from the species Alloprevotella rava.

Optionally, the or each bacterial species is selected from the genus Prevotella and is selected from one or more of the species:
(i) Prevotella nigrescens;
(ii) Prevotella oris;
(iii) Prevotella pallens;
(iv) Prevotella sp. oral taxon 299 str. F0039;
(v) Prevotella nanceinsis; and
(vi) Prevotella nigrescens.

Preferably, the or each bacterial species is selected from the genus Prevotella and is selected from the species Prevotella nigrescens

Optionally, the or each bacterial species is selected from the genus Prevotella_6 and is selected from the species Prevotella_6 salivae.

Optionally, the or each bacterial species is selected from the genus Prevotella_7 and is selected from one or more of the species:
(i) Prevotella_7 sp. oral clone GI059;
(ii) Prevotella_7 jejuni;
(iii) Prevotella_7 melaninogenica; and
(iv) Prevotella_7 HMT 306

Optionally, the or each bacterial species is selected from the genus Fusobacterium and is selected from the species Fusobacterium necrophorum ssp. Necrophorum.

Optionally, the or each bacterial species is selected from the genus Treponema and is selected from the species Treponema sp. 5:22:BH022.

Preferably, the or each bacterial species is selected from the genus Treponema and is selected from the species Treponema 2 HMT 263.

Preferably, the or each bacterial species is selected from the genus Streptococcus and is selected from one or more of the species:
(i) Streptococcus australis;
(ii) Streptococcus infantis;
(iii) Streptococcus sanguinis;
(iv) Streptococcus parasanguinis; and
(v) Streptococcus oralis.

Further preferably or additionally, the or each bacterial species is selected from the genus Neisseria and is selected from one or more of the species:
(i) Neisseria mucosa;
(ii) Neisseria pharynges;
(iii) Neisseria perflava; and

Further preferably or additionally, the or each bacterial species is selected from the genus Haemophilus and is selected from the species Haemophilus parainfluenzae.

Further preferably or additionally, the or each bacterial species is selected from the genus Veillonella and is selected from one or more of the species:
(i) Veillonella dispar;
(ii) Veillonella parvula;
(iii) Veillonella rogosae; and
(iv) Veillonella atypica.

Further preferably or additionally, the or each bacterial species is selected from the genus Granulicatella; and is selected form the species: elegens.

Further preferably or additionally, the or each bacterial species is selected from the genus Gemella and is selected from one or more of the species:
(i) Gemella haemolysans; and
(ii) Gemella sanguinis.

Further preferably or additionally, the or each bacterial species is selected from the genus Streptococcus and is selected from one ore more of the species Streptococcus parasanguinis.

Further preferably or additionally, the or each bacterial species is selected from the genus Prevotella_7 and is selected from one or more of the species:
(i) Prevotella_7 jejuni; and
(ii) Prevotella_7 melaninogenica.

Further preferably or additionally, the or each bacterial species is selected from the genus Prevotella and is selected from the species Prevotella nanceiensis.

Further preferably or additionally, the or each bacterial species is selected from the genus Selenomonas and is selected from the species Selenomonas sputigena.

Further preferably or additionally, the or each bacterial species is selected from the genus Megasphaera and is selected from the species Megasphaera micronuciformis.

Further preferably or additionally, the or each bacterial species is selected from the genus Prevotella_6 and is selected from the species Prevotella_6 salivae.

Preferably, the or each bacterial species is selected from one or more of the species:
(i) Streptococcus australis;
(ii) Streptococcus infantis;
(iii) Streptococcus sanguinis;
(iv) Streptococcus parasanguinis;
(v) Streptococcus oralis.
(vi) Neisseria mucosa;
(vii) Neisseria pharynges;
(viii) Neisseria perflava;
(ix) Haemophilus parainfluenzae;
(x) Veillonella dispar;
(xi) Veillonella parvula;
(xii) Veillonella rogosae;
(xiii) Granulicatella elegens;
(xiv) Gemella haemolysans;
(xv) Gemella sanguinis;
(xvi) Streptococcus parasanguinis;
(xvii) Prevotella_7 jejuni;
(xviii) Prevotella_7 melaninogenica;
(xix) Prevotella nanceiensis;
(xx) Selenomonas sputigena;
(xxi) Megasphaera micronuciformis; and
(xxii) Prevotella_6 salivae.

Optionally, the or each bacterial species is selected from the genus Prevotella and is selected from one or more of the species:
(i) Prevotella nigrescens;
(ii) Prevotella oris; and
(iii) Prevotella sp. oral taxon 299 str. F0039.

Optionally or additionally, the or each bacterial species is selected from the genus Treponema and is selected from the species Treponema sp. 5:22:BH022.

Optionally or additionally, the or each bacterial species is selected from the genus Prevotella_7 and is selected from one or more of the species Prevotella_7 sp. oral clone GI059.

Optionally or additionally, the or each bacterial species is selected from the genus Alloprevotella and is selected from the species Alloprevotella tannerae.

Optionally or additionally, the or each bacterial species is selected from the genus Fusobacterium and is selected from the species Fusobacterium necrophorum ssp. Necrophorum.

Optionally, the or each bacterial species is selected from one or more of the species:
(i) Prevotella nigrescens;
(ii) Prevotella oris;
(iii) Prevotella sp. oral taxon 299 str. F0039;
(iv) Treponema sp. 5:22:BH022;
(v) Prevotella_7 sp. oral clone GI059;
(vi) Alloprevotella tannerae; and
(vii) Fusobacterium necrophorum ssp. Necrophorum.

Optionally, the or each bacterial species is selected from the genus Megasphaera and is selected from the species Megasphaera micronuciformis.

Optionally or additioanlly, the or each bacterial species is selected from the genus Prevotella and is selected from one or more of the species:
(i) Prevotella pallens; and
(ii) Prevotella sp. oral taxon 299 str. F0039.

Optionally, the or each bacterial species is selected from one or more of the species:
(i) Megasphaera micronuciformis;
(ii) Prevotella pallens; and
(iii) Prevotella sp. oral taxon 299 str. F0039.

Optionally, the or each bacterial species is selected from the genus Alloprevotella and is selected from the species Alloprevotella tannerae.

Preferably, the or each bacterial species is selected from the genus Alloprevotella and is selected from the species Alloprevotella tannerae ASV 1.

Preferably, the or each bacterial species is selected from the genus Alloprevotella and is selected from the species Alloprevotella tannerae ASV 2.

Optionally or additionally, the or each bacterial species is selected from the genus Neisseria and is selected from one or more of the species:
(i) Neisseria mucosa; and
(ii) Neisseria pharynges.

Optionally, the or each bacterial species is selected from one or more of the species:
(i) Alloprevotella tannerae;
(ii) Neisseria mucosa; and
(iii) Neisseria pharynges.

Further preferably, the or each bacterial species is selected from the genus Porphyromonas and is selected from the species Porphyromonas endodontalis.

Further preferably or additionally, the or each bacterial species is selected from the genus Bergeyella and is selected from the species Bergeyella HMT 206.

Further preferably or additionally, the or each bacterial species is selected from the genus Rothia and is selected from the species Rothia mucilaginosa.

Further preferably or additionally, the or each bacterial species is selected from the genus Schaalia and is selected from the species Schaalia lignae.

Further preferably or additionally, the or each bacterial species is selected from the genus Schaalia and is selected from the species Schaalia HMT 180.

Further preferably or additionally, the or each bacterial species is selected from the genus Solobacterium and is selected from the species Solobacterium moorei.

Optionally, the sample of the microbiome is selected from whole blood, serum, plasma, urine, interstitial fluid, peritoneal fluid, cervical swab, tears, saliva, buccal swab, skin, brain tissue, and cerebrospinal fluid.

Optionally, the sample of the microbiome is a sample of the oral microbiome.

Optionally, the sample of the oral microbiome is selected from saliva, and buccal swab.

Optionally, the sample of the oral microbiome is a sample of the oral cavity.

Optionally, the sample of the oral microbiome is a saliva sample.

Preferably, the method comprises the step of (a) providing a saliva sample of the oral microbiome from the subject.

Optionally, the quantitative level or presence of the one or more bacterial species in the sample is indicative of depression in a subject.

Further optionally, the presence of the one or more bacterial species in the sample is indicative of depression in a subject.

Further optionally, the presence of all of the bacterial species in the sample is indicative of depression in a subject.

Further optionally, the quantitative level of the one or more bacterial species in the sample is indicative of depression in a subject.

Further optionally, the quantitative level of all of the bacterial species in the sample is indicative of depression in a subject.

Optionally, the method is an in vitro method.

### BRIEF DESCRIPTION OF THE DRAWINGS

Embodiments of the invention will be described with reference to the following non-limiting examples and the accompanying drawings in which:

FIG. 1 illustrates (a) community composition shown by the relative abundance of prevalent phyla; and (b) community composition shown by the relative abundance of prevalent families;

FIG. 2 illustrates Canonical Correspondence Analysis (CCA), which relates samples (dots) to significant environmental variables (arrows), wherein the variation that can be explained by each axis is significant (smoking: p=0.001, depression: p=0.005);

FIG. 3 illustrates (a) differential abundance analysis that identified 12 ASVs (that cover 10 genera and 3 phyla) that had an altered abundance in the depressed cohort compared to controls; (b) network of statistically significant pairwise microbial interactions unique to the depressed cohort, wherein nodes are bacterial species, edges are interactions (green: positive co-occurrence, red: negative co-exclusion), and opportunistic pathogens are highlighted in red; and (c) inferred metagenomic content analysis identified functional changes in depressed subjects;

FIG. 4 illustrates measuring the predictive power of Set A* for identifying depression from a saliva sample;

FIG. 5 illustrates measuring the predictive power of Set B* for identifying depression from a saliva sample;

FIG. 6 illustrates measuring the predictive power of Set A for identifying depression from a saliva sample;

FIG. 7 illustrates measuring the predictive power of Set B for identifying depression from a saliva sample;

FIG. 8 illustrates species diversity of salivary samples differs between depression and healthy cohorts;

FIG. 9 illustrates the structure of the oral microbiome in subjects with depression is subtly different compared with controls; and

FIG. 10 illustrates the abundance of amplicon sequence variants is significantly different in the depressed cohort.

### EXAMPLES

### Materials and Methods

### Study Design and Sample Collection

Samples were utilised from the Ulster University Student Wellbeing Study (UUSWS), conducted as part of the World Health Organisation (WHO) World Mental Health International College Student (WMH-ICS) Project. Ethical approval was obtained from Ulster University Research Ethics Committee (REC/15/0004). First year students were recruited during registration where they gave written consent, provided a saliva sample, and were given a unique, anonymous number to complete an online mental health survey clinically validated according to the Diagnostic and Statistical Manual of Mental Disorders, Fifth Edition (DSM-IV).

### Survey Responses

The survey instrument was adapted from the WMH Composite International Diagnostic Interview (CIDI) version 3.0. Life-time depression is determined based on the response to seven questions (Likert scale) corresponding to DSM-IV criteria for depression. To calculate lifetime major depressive disorder (LT-MDE), the first six symptoms/questions were recoded to; 4=“all or most of the time” and 0=‘none of the time’, and summed. If at least one of the first four symptoms was “all or most of the time” and the sum of all six symptoms was at least 15 then participants met the criteria for depression.

### Case Selection

Cases (n=40) were selected from participants who met the criteria for LT-MDE. Healthy controls were individuals with no history of mental health problems (n=43), closely matched to cases by age and gender and, where possible, by smoking status (Table 1). There was no significant difference in age (p=0.16) or gender composition (p=0.8) between the case and control groups. Gender and smoking were all included as potential confounders for microbiome composition in downstream analyses.

### Sample Collection

Saliva samples (passive drool) were collected using Oragene™ OG-500 kits (DNA Genotek, Ontario Canada), enabling self-collection and stabilisation of DNA at room temperature. Participants were not to comsume any food or drink, except water, for at least 30 mins prior to sample collection. Cases of depression (n=43) were selected based on survey responses to seven questions corresponding to DSM-IV criteria for depression using a Likert scale response, and controls were matched where possible for age, gender, ethnicity and smoking status (see Table 1).

TABLE 2
Sample demographics: Age, gender, smoking status and depression
severity score based on participant response to Composite International
Diagnostic Interview (CIDI) depression section. Maximum depression
score for inclusion in healthy group = 15, and minimum depression
score for inclusion in depression group = 30.

| | | |
|---|---|---|
| Demographic | Controls (n = 43) | Cases (n = 44) |

| Age (mean) | 21 | | 22 | |
|---|---|---|---|---|
| (Range ± SD) | (18-36 ± 3.9) | | (18-38 ± 5.3) | |
| Gender | | | | |
| Male | 13 | (30.2) | 11 | (25.0) |
| Female | 30 | (69.8) | 33 | (75.0) |
| Smoking status | | | | |
| Past (%) | 0 | (0.0) | 7 | (29.5) |
| Occasional (%) | 6 | (14.0) | 9 | (20.5) |
| Daily (%) | 3 | (7.0) | 13 | (29.5) |
| Never (%) | 34 | (79.1) | 11 | (25.0) |
| Missing (%) | | | 4 | (9.12) |

| | | |
|---|---|---|
| Depression score (mean) | 34.6 | 10.1 |
| (Range ± SD) | (32-35 ± 0.9) | (7-14 ± 2.5) |

### Microbial DNA Extraction and Sequencing

Microbiome DNA purification was carried out using MasterPure™DNA Purification Kit and Ready-Lyse™Lysozyme from the MasterPure™ Gram Positive DNA Purification Kit (Epicentre, Madison, US) according to the manufacturer's instructions. The quantity of DNA was measured on a Nanodrop spectrometer (Fisher Scientific, Loughborough, UK) and the quality measured using the 260/280 ratio and 1.5% gel electrophoresis. To confirm the presence of bacterial DNA, broad range 16S PCR was carried out. Finally, 50 μl of 22 ng/μl of good quality DNA was sent to The Forsyth Institute for 16S next generation sequencing, using the HOMINGS technique for microbial analysis.

To begin sequencing, PCR amplification of 10-50 ng of sample DNA was carried out using V3-V4 primers and 5 PrimeHot Master Mix. The amplicon product was then purified using Solid Phase Reversible Immobilization with AMPure beads, and 100 ng of each amplicon library was pooled, gel-purified, and quantified using a bioanalyser and subsequent qPCR. Finally, 12 pM of the library mixture was then spiked with 20% PhiX (Illumina, San Diego, CA), and sequenced on Illumina MiSeq.

### Bioinformatics Processing

Sequences were denoised with the R v3.4.2 package dada2 (v1.4.0) using a standard operating protocol. In brief, quality filtered paired end sequences reads were trimmed, denoised, and joined into contigs. Chimeric sequences were removed and taxonomy was assigned to the denoised sequence reads using the Ribosome Database Project's naïve Bayesian classifier and the SILVA 16S rRNA gene reference database. The denoised sequences represented exact 16S rRNA gene sequence variants. These sequence variants were not binned into fuzzy operational taxonomic units as the amplicon sequence variant (ASV) paradigm is superior to a sequence similarity cutoff approach. A de novo phylogenetic tree was generated from the ASV with the R package phangorn v2.3.1. The abundance of 16S rRNA gene sequence variants, taxonomy data, phylogenetic tree, and sample information (e.g. depression status) were combined into a phyloseq v1.20.0 object for statistical analysis. ASVs of interest were further analysed (e.g. differentially ASVs) by matching sequences against the Human Oral Microbiome Database; ASVs were matched to a species level to identify possible mechanisms of action.

### Statistical Analysis

The microbial community composition (b-diversity) was estimated using Bray-Curtis dissimilarity with the R package vegan (v2.4.3). The Bray-Curtis dissimilarity was estimated from normalised copy number compensated microbiome census data. To detect statistical differences in b diversity between groups we used permutational multivariate analysis of variance (PERMANOVA) implemented in the vegan package. A b-dispersion test (vegan::betadisper) was used to verify that statistically significant groups identified by PERMANOVA had the same dispersions. The community structure of the oral microbiome was visualised with a canonical correspondence analysis (CCA) biplot; statistically significant environmental terms (determined by the PERMANOVA test) were included on the ordination. The significance of the CCA ordination solution was confirmed with a permutation test (vegan::anova.cca).

Differential abundance of ASVs was tested using the R package DESeq2 package (v1.18.1). To preserve statistical power very rare ASVs (present in less than 10% of samples) were removed prior to testing. DESeq2 implements a generalised linear model (GLM) based on the negative binomial distribution to detect differential expression in count data while accounting for differences in library size and biological variation. Although DESeq2 was originally developed for RNASeq data recent work has shown that it is well suited for application to microbiome census data compared with other widely used statistical techniques that rely on destructive normalisation techniques. Raw reads from both the microbiome census data and functional profiles were fitted to a negative binomial GLM and a Wald test was used to determine the significance of GLM coefficients.

DESeq2 corrects for multiple testing with the Benjamini-Hochberg adjustment; statistical significance was determined at the 5% level. Differential abundance was expressed as log₂ fold change in depressed subjects relative to control subjects. Differential abundance was determined for both microbiome census data and functional profiles with a design blocking variation introduced by smoking and gender (i.e. only considering the potential effects of depression on abundance).

The SparCC algorithm implemented in the fastspar (v0.0.3) software package was used to calculate the correlation (co-occurrence) of ASVs. The co-occurrence matrix is a symmetrical N×N matrix (where N gives the total number of ASVs). Exact p-values were calculated for the co-occurrence matrix via permutation tests (1000 iterations). A correlation matrix and exact p-value matrix were estimated for non-smoking depressed subjects and non-smoking healthy subjects. The p-value matrix was false discovery rate adjusted. The R package igraph (v1.1.2) was used to build an undirected graph from each co-occurrence matrix, in which nodes are ASVs and edges are the interaction type (e.g. co-presence or co-absence). Edges with p>0:05 were removed and nodes with no edges after filtering were also removed. This resulted in a graph subset for both the healthy and depressed cohort. The set difference of the graphs was taken to identify statistically significant microbial interactions that were unique to the depressed cohort.

16S rRNA Gene Copy Number Compensation and Prediction of Metagenomic Content with PICRUSt

Different bacteria have a different amount of 16S rRNA gene copies (16S copy number), which can bias estimates of abundance and diversity (a bacteria with a very high 16S copy number will have an artificially inflated abundance). The 16S copy number of ASVs was estimated from the ribosomal RNA database (v5.1). Approximately 50% of the ASVs were not present in the database. The copy number for unknown ASVs was estimated using the copy number of the known DSVs and a phylogenetic ancestral state reconstruction algorithm (the R package picante 1.6-2). The compensated abundance an ASV Y_{i;j} was calculated by Y_{i;j}=X_{i;j}/Zᵢ where X_{i;j} gives the count of the i-th amplicon sequence variant from the j-th sample, and Zᵢ gives the copy number. ASVs with an abundance less than 1 for every sample after this transformation were removed. The compensated counts were used for every stage of the analysis, except differential abundance testing and functional prediction.

PICRUSt was used to identify differences to inferred functional content between depressed and control groups. In brief: ASVs were added to the GreenGenes version 13.5 reference database. ASVs that diverged by more than 3% were discarded according to a standard operating protocol. New PICRUSt precalculated files were created from the new reference database. ASV abundance was normalised by 16S copy number and the bacterial composition was used to predict KEGG orthologs (KO) from the new precalculated files. KOs were collapsed into KEGG pathways using the categorize_by_function.py command provided by PICRUSt. Linear discriminate analysis effect size (LEfSe) was used to identify differentially abundant functional pathways in the depressed cohort.

### Stratification by Depression Status

The kohonen package (v3.0.485) in R was used to implement a Super Organising Map (SOM) with separate layers for each data type. Our rationale for using a SOM was that it is a multimodal data-driven algorithm. Data driven approaches make no assumptions input data. Microbiome census data are typically highly dimensional, sparse, compositional, and have an uneven mean-variance relationship; all of which can be problematic for standard models. It is common for different types of sensors or sensors to record information about subjects in an experiment. Each information acquisition framework is termed a modality.

Briefly, multimodal classification combines input data from different modalities to gain a global view of the modelled system. This concept complements modelling the microbiome, which is a holistic system that “refers to the entire habitat, including the microorganisms, their genomes, and the surrounding environmental conditions”. The SOM was used to perform two-class supervised classification (healthy versus depressed). Three types of microbiome data were used to train the SOM (four including class memberships): untransformed raw microbiome census data, the library size for each sample, and environmental data. Our motivation for using unnormalised data is that there is no universally accepted normalisation approach and each normalisation method is associated with different drawbacks. For example, rarefaction discards data which causes information loss and proportions can be distorted by highly abundant species. By combining raw microbiome census data with the library size in this multivariate analysis bias can be mitigated without negative side effects introduced by normalisation. The cohort was randomly divided into a training set (80% of samples) and a testing set (20% of samples). After training the first three layers of the SOM were used to predict the class of unseen data. The predictions were compared against the true class memberships to evaluate the performance of the model.

### Example 1

### The Composition and Structure of the Oral Microbiome Differs Between Depression and Healthy Cohorts

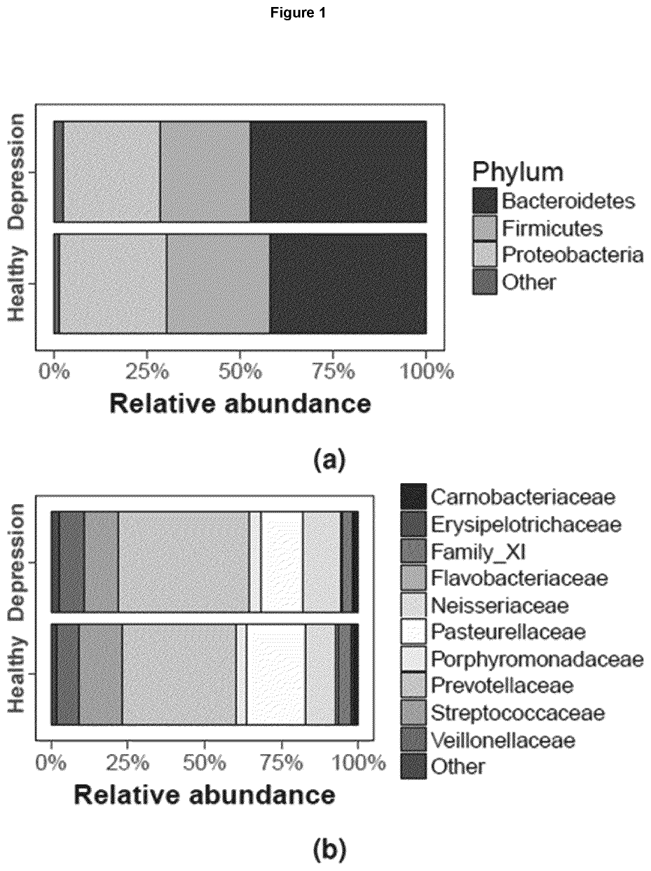

Sequencing the V3-V4 regions of the 16S rRNA gene generated a total of approximately 12.5 million sequence reads (median±MAD): _66,000_28,000 sequence reads per subject, and the denoised dataset contained 2883 unique sequences covering 9 phyla, 18 classes, 33 orders, 53 families, 84 genera, and 133 species. The dominant phyla present in the oral microbiota across both cohorts were Bacteroidetes (42.18±13.87%), Proteobacteria (24.57±17.29%), and Firmicutes (26.62±9.93%) (top panel of FIG. 1a). The most prevalent families in the oral microbiota for all subjects were Prevotellaceae (37.22%), Pasteurellaceae (15.60%), Streptococcaceae (10.59%), Veillonellaceae (5.46%), and Neisseriaceae (5.50%) (FIG. 1b).

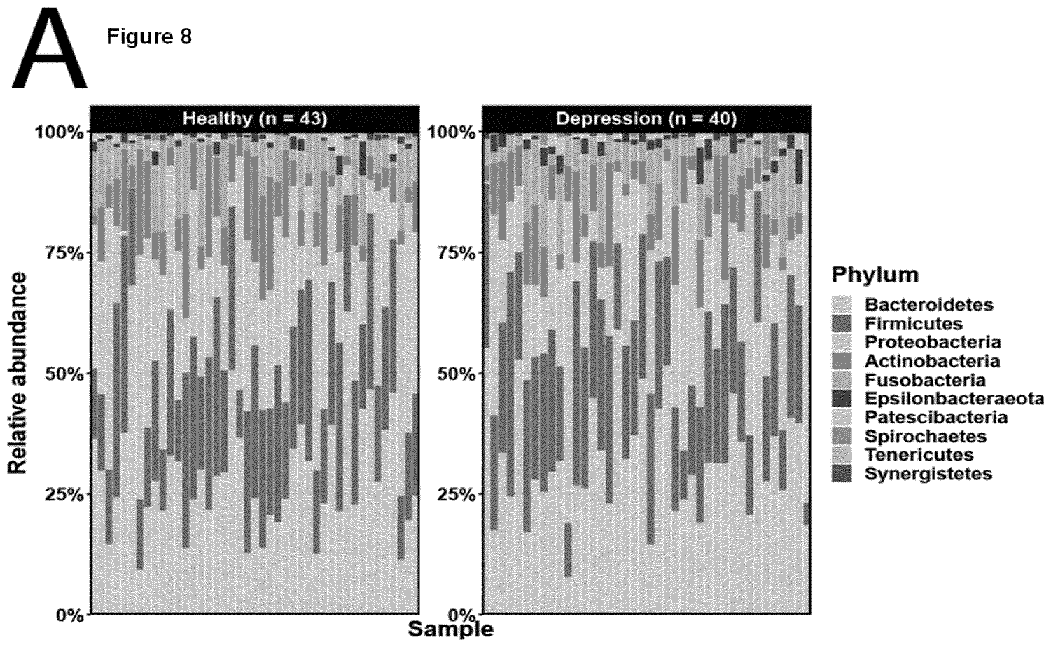

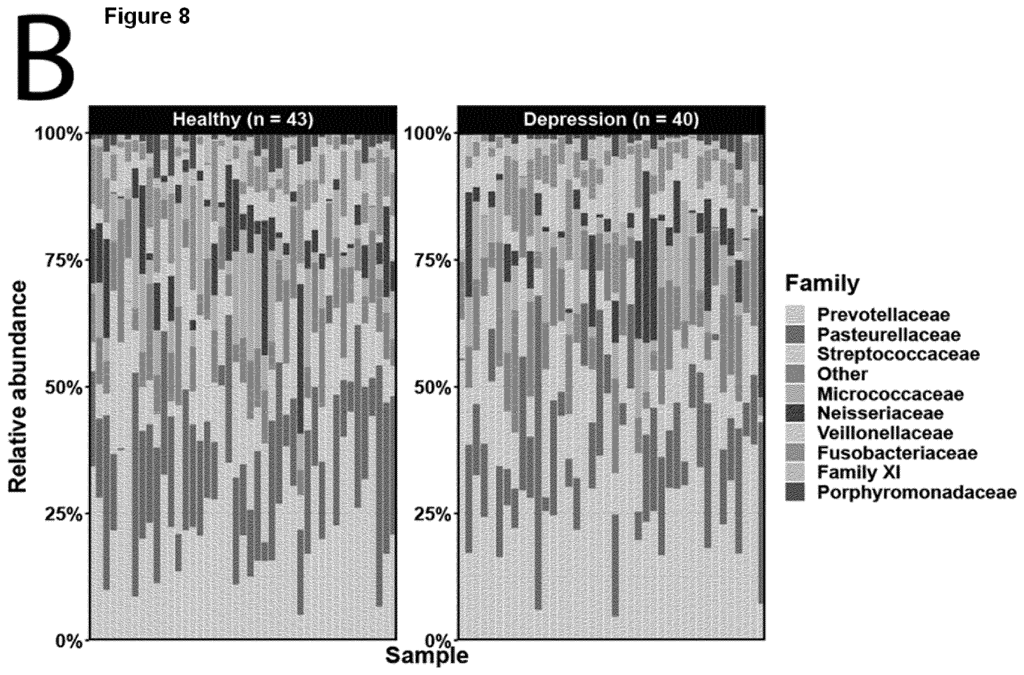

A further analysis corroborated our findings after sequencing the V3-V4 regions of the 16S rRNA gene generated approximately 12.5 million sequences (median±MAD): ˜66,000±28,000 sequence reads per subject, and the denoised dataset contained 3613 unique sequences covering phyla, 19 classes, 42 orders, 75 families, 144 genera, and 181 identified species. The dominant phyla present in the salivary microbiome across both cohorts were Bacteroidetes (29.6±11.8%), Firmicutes (24.5±9.3%), and Proteobacteria (21.2±9.3%) (FIG. 8A). The most prevalent families in the salivary microbiome for all subjects were Prevotellaceae (26.9±10.8%), Pasteurellaceae (16.4±10.3%), and Streptococcaceae (9.9±5.3%) (FIG. 8B).

FIG. 8 shows the overall composition of the oral microbiome matches previous reports in the literature: Community composition shown by relative abundance of prevalent (A) phyla and (B) families. Taxa present in fewer that 5% of samples and with a relative abundance smaller than 0.1% were removed.

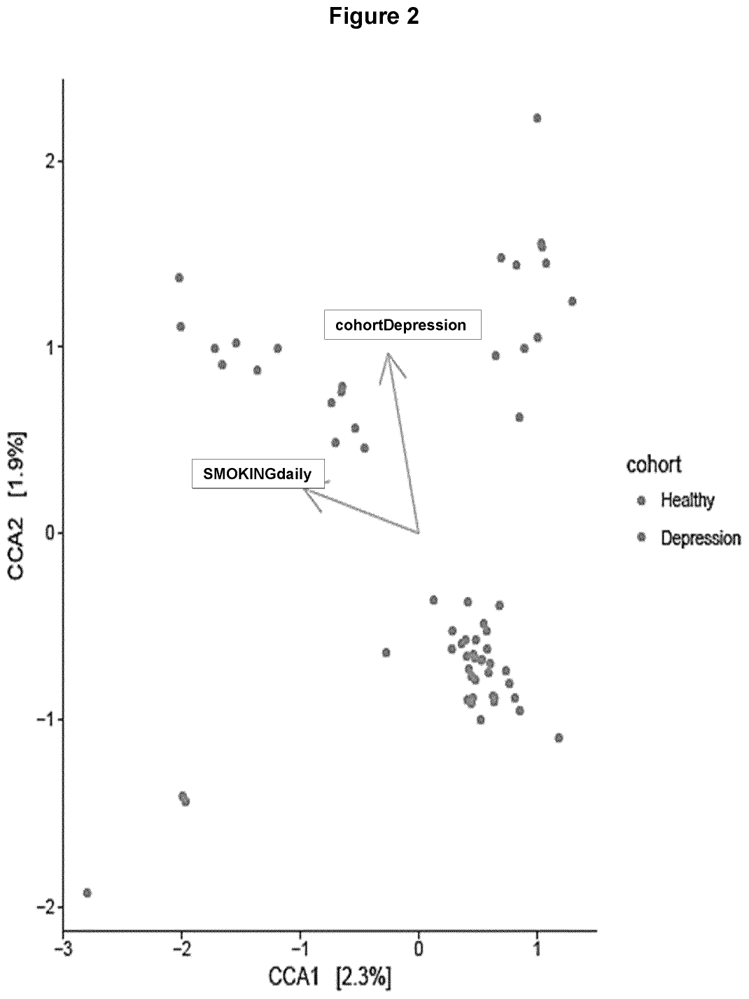

The structure and composition of the oral microbiome was characterised with a range of techniques, beginning with ecological measures such as richness (the number of unique ASVs present in a sample), alpha diversity and beta diversity. Alpha diversity began with simple estimators such as the Shannon diversity index and the Inverse Simpson diversity index, and then moved on to non-parametric species estimators such as the Abundance-based coverage estimator (ACE) and Chaol which provide a measure of richness while compensating for differing sampling intensity across samples. Faith's Phylogenetic Diversity index was used to measure richness while incorporating data about phylogenetic relationships. Depression was not associated with significant changes to richness or alpha diversity for any of the tested metrics. The Bray-Curtis dissimilarity statistic was used to measure beta diversity, and significant differences were found in the composition of the oral microbiota between depression and control groups (PERMANOVA: p=0.038). Smoking was also associated with significant differences in composition of the oral microbiota (PERMANOVA: p<0.001). Canonical Correspondence Analysis (CCA) was used to test and visualise the affect that statistically significant environmental variables had on the structure of the oral microbiota. The CCA biplot shows clear clustering between depressed and healthy cohorts into distinct groups, also clustering between smokers and non-smokers (see FIG. 2).

The first canonical axis was negatively correlated with smoking daily, and the second canonical axis was positively correlated with depression and slightly positively correlated with smoking daily. Smoking did affect microbiome composition, however affects were opposing to cohort differences, suggesting the cohort separation is not an artefact of smoking status.

### Example 2

Microbial Abundance and Interactions were Significantly Different in the Depressed Cohort

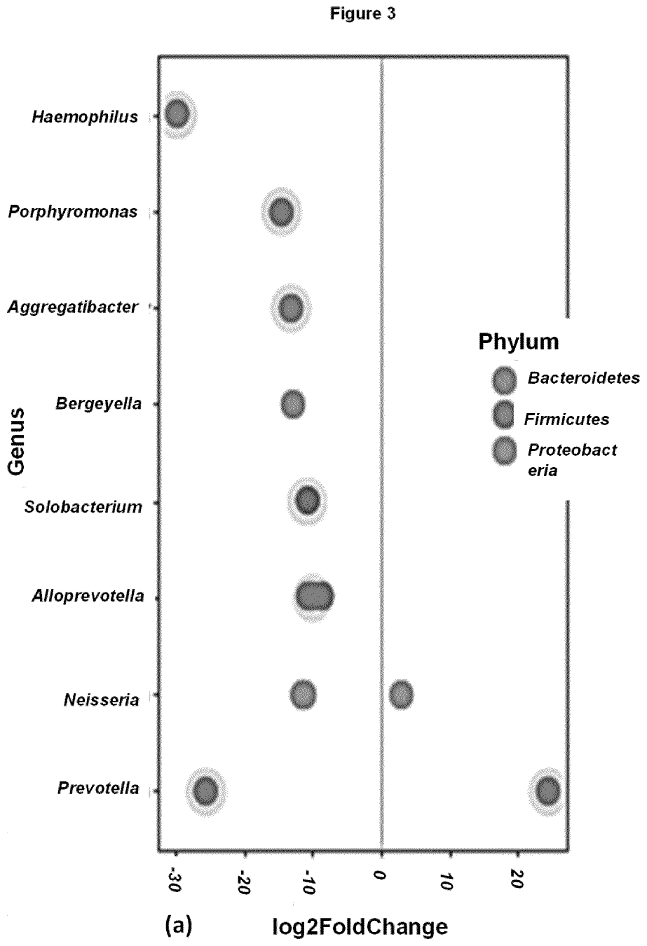

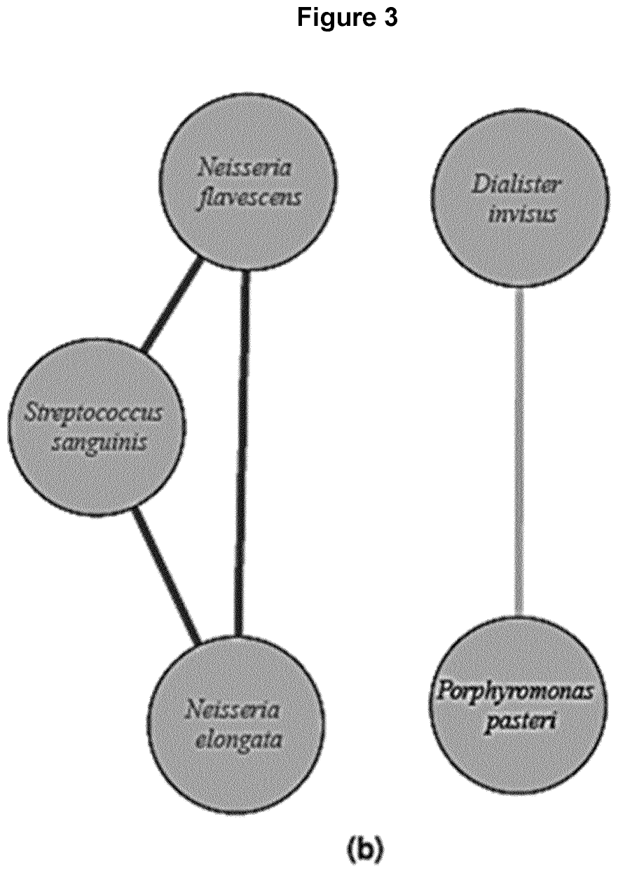

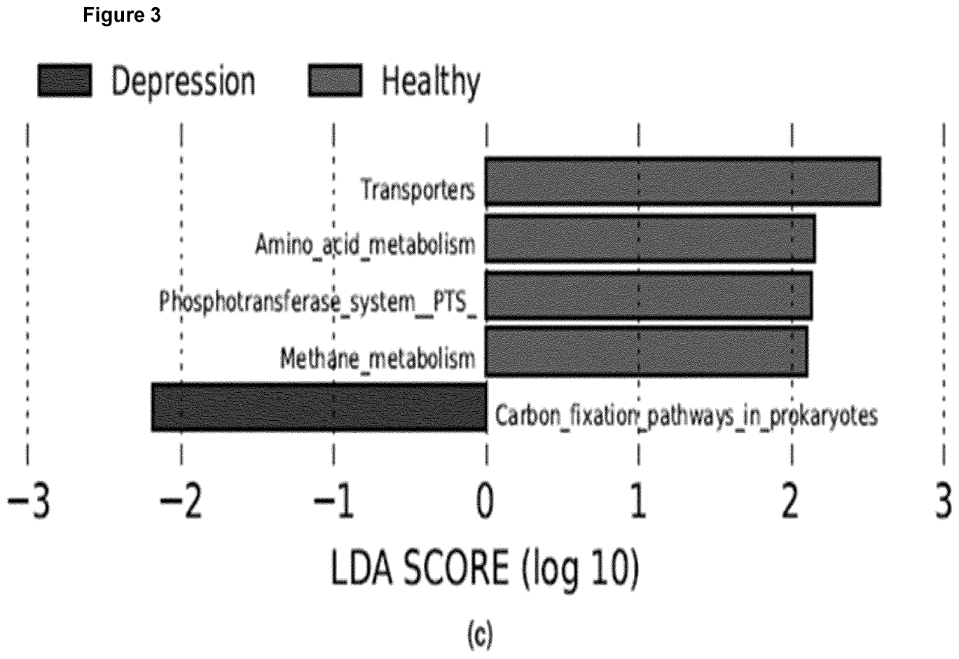

Differential abundance testing of prevalent ASVs found that 12 bacterial species were differentially abundant in the depressed cohort relative to the controls (see FIG. 3a). From these sequence variants, 2 were significantly more abundant in depressed subjects, and 10 were significantly less abundant in depressed subjects. These differentially abundant sequences were matched against the Human Oral Microbiome Database in order to gain an understanding of possible underlying mechanisms of action.

The majority of identified organisms were opportunistic pathogens (i.e. under normal conditions they are commensal) or normal commensal organisms. Opportunistic pathogens that are decreased in depression have been associated with endodontic infections, halitosis, infective endocarditis, and pulpal pathogens. Opportunistic pathogens that have been found to be increased in depression include P. nigrescens and N. sicca. P. nigrescens is associated with periodontitis, whilst N. sicca is a commensal pathogen.

Inferred metagenomic content analysis (PICRUSt and LEfSe) was used to identify possible functional changes in the oral microbiome of depressed subjects. These observed changes include a decrease in carbon fixation pathways and increases in amino acid metabolism, methane metabolism, transporters, and phosphotransferase system (see FIG. 3c). An analysis of microbial interactions from estimated microbial co-occurrence patterns found a group of statistically significant interactions unique to the depressed cohort (see FIG. 3b). A co-exclusion relationship was found between Neisseria flavescens, Streptococcus sanguinis, and Neisseria elongata in the depressed cohort; a co-presence relationship was found between Dialister invisus and Porphyromonas pasteri.

### Example 3

### Observed Alterations Enabled Accurate Stratification of Depression Status

To determine if the observed microbiome alterations were significant enough for stratification of depression status a multimodal data-driven supervised learning classification algorithm called a Super Organising Map (SOM) was applied to the microbiome census data (see Methods). The classification task was to distinguish between control and depressed subjects (two-class classification). Models were trained on 80% of the data. The generalisation ability of the models was validated by making predictions on unseen data (the remaining 20%). To measure the performance of the classification models a variety of metrics were used, including balanced accuracy, positive predictive value (PPV), and negative predictive value (NPV). A multimodal SOM was able to predict depression with a balanced accuracy of 83.35% on unseen data (see Table 2).

TABLE 2
Performance of classification algorithms applied for
depression prediction from microbiome census data.

| Model | Accuracy | Sensitivity | Specificity | PPV | NPV |
|---|---|---|---|---|---|
| SOM (oral) | 82.35% | 66.77% | 100.00% | 1.00 | 0.73 |
| PLS-DA (gut) | 66.50% | 86.00% | 47.00% | Not reported | Not reported |
| Random Forest (gut) | Not reported | Not reported | Not reported | Not reported | Not reported |

### Example 4

### Set a*: 13 Bacterial Groups

Each bacterial group is an amplicon sequence variant. The amplicon sequence variants are a denoised 16S DNA sequence around 200 nucleotides long. These DNA sequences are mapped to taxonomic databases. This is done to give the sequences a name that humans can understand (e.g. E. coli). Different amplicon sequence variants can be mapped to the same genus (e.g. Prevotella) but are distinct entities. Some—but not all—amplicon sequence variants can be mapped to a species level (see Table 3).

TABLE 3
Bacterial groups in Set A*.

| Genus | Species |
|---|---|
| Prevotella | |
| Prevotella | |
| Haemophilus | |
| Bergeyella | |
| Porphyromonas | |
| Aggregatibacter | |
| Alloprevotella | tannerae |
| Neisseria | |
| Solobacterium | |
| Alloprevotella | |
| Neisseria | mucosa/pharynges |
| Neisseria | |
| Alloprevotella | |

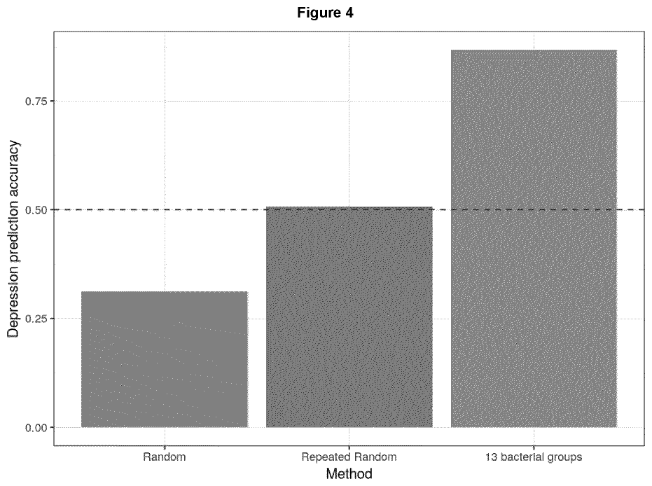

FIG. 4 demonstrates that Set A* (13 bacterial groups) provided an unexpected technical benefit when predicting depression. Depression was predicted using a random set of bacterial groups (n=13, see left bar on FIG. 4). Depression was also predicted using 100 random sets of bacterial groups (n=13, see middle bar on FIG. 4). Both approaches performed worse or equivalent to random chance (50% accuracy represented by red dotted line on FIG. 4. The invention disclosed herein delivered an unexpected technical benefit (right bar on FIG. 4).

### Example 5

### Set B*: 27 Bacterial Groups

A specific combination of 27 amplicon sequence variants was identified using aggregating ensemble feature selection. This specific combination of bacterial groups can predict depression with 79% accuracy (see Table 4).

TABLE 4
Bacterial groups in Set B*.

| Genus | Species |
|---|---|
| Streptococcus | australis/infantis/ |
| | sanguinis |
| Neisseria | mucosa/perflava/ |
| | subflava |
| Haemophilus | parainfluenzae |
| Veillonella | dispar |
| Granulicatella | elegans |
| Streptococcus | |
| Prevotella | |
| Haemophilus | |
| Megasphaera | |
| Gemella | haemolysans/ |
| | sanguinis |
| Streptococcus | parasanguinis |
| Prevotella_7 | jejuni/ |
| | melaninogenica |
| Selenomonas_3 | |
| Gemella | sanguinis |
| Streptococcus | oralis/ |
| | parasanguinis |
| Porphyromonas | |
| Porphyromonas | |
| Haemophilus | parainfluenzae |
| Prevotella_7 | melaninogenica |
| Veillonella | parvula/rogosae |
| Prevotella | nanceiensis |
| Veillonella | |
| Selenomonas | sputigena |
| Veillonella | rogosae |
| Megasphaera | micronuciformis |
| Prevotella_6 | salivae |
| Veillonella | |

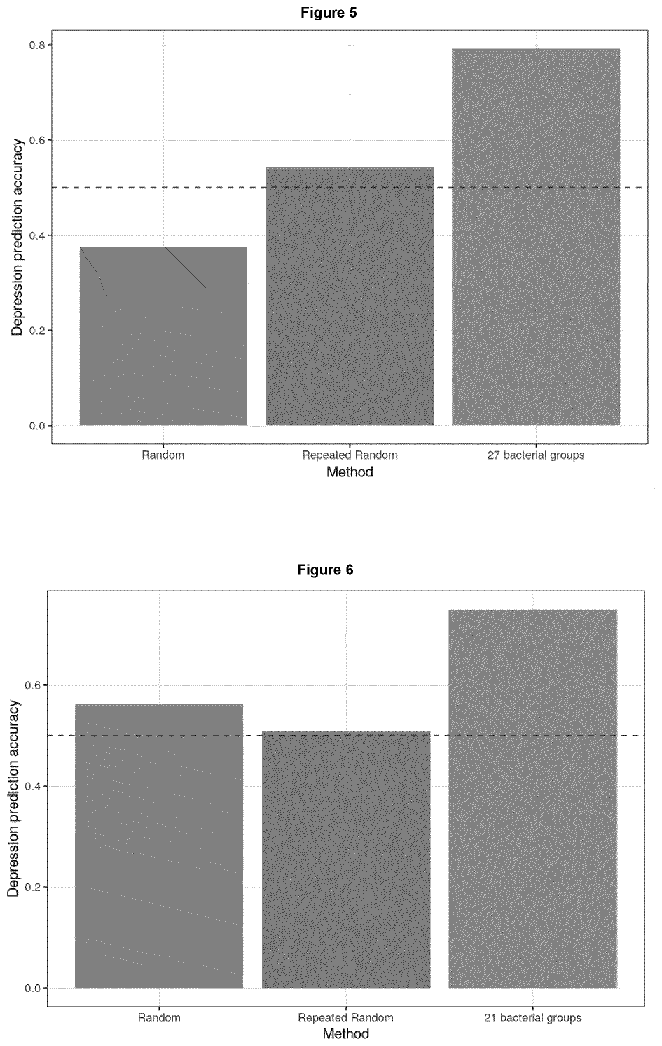

Depression was predicted using a random set of bacterial groups (n=27, see left bar on FIG. 5). Depression was also predicted using 100 random sets of bacterial groups (n=27, see middle bar on FIG. 5). Both approaches performed worse or equivalent to random chance (50% accuracy represented by red dotted line on FIG. 5). This aspect of the disclosed invention delivered an unexpected technical benefit (right bar on FIG. 5).

### Example 6

### Set A: 21 Bacterial Groups

A set of 21 bacterial groups that are differentially abundant in a depressed cohort were identified. The predictive power of these 21 bacterial groups was measured. Depression was predicted with 75% accuracy (see Table 5).

TABLE 5
Bacterial groups in Set A.

| Genus | Species |
|---|---|
| Prevotella | Prevotella nigrescens |
| Haemophilus | NA |
| Prevotella | Prevotella sp. oral taxon 299 str. F0039 |
| Rothia | NA |
| Treponema 2 | Treponema sp. 5:22:BH022 |
| Prevotella 7 | Prevotella sp. oral clone GI059 |
| Bergeyella | uncultured bacterium |
| Porphyromonas | NA |
| Actinomyces | NA |
| Actinomyces | NA |
| Neisseria | uncultured bacterium |
| Alloprevotella | Alloprevotella tannerae |
| Prevotella | Prevotella oris |
| Solobacterium | NA |
| Alloprevotella | Alloprevotella tannerae |
| Alloprevotella | NA |
| Neisseria | uncultured bacterium |
| Leptotrichia | NA |
| Fusobacterium | Fusobacterium necrophorum subsp. necrophorum |
| Neisseria | uncultured bacterium |
| Veillonella | NA |

Depression was predicted using a random set of bacterial groups (n=21, see left bar on FIG. 6) Depression was also predicted using 100 random sets of bacterial groups (n=21, see middle bar on FIG. 6). Both approaches performed worse or equivalent to random chance (50% accuracy represented by red dotted line on FIG. 6). This aspect of the disclosed invention delivered an unexpected technical benefit (right bar on FIG. 6).

### Example 7

### Set B: 35 Bacterial Groups

A specific combination of 35 amplicon sequence variants (bacterial groups) was identified using aggregating ensemble feature selection. Depression was predicted with up to 85.8% accuracy (see Table 6).

TABLE 6
Bacterial groups in Set B.

| Phylum | Genus | Species | Phylum | Genus | Species |
|---|---|---|---|---|---|
| Firmicutes | Selenomonas 3 | NA | Firmicutes | Lachnoanaerobaculum | NA |
| | | | Proteobacteria | Neisseria | NA |
| Proteobacteria | Haemophilus | NA | Firmicutes | Streptococcus | NA |
| Actinobacteria | Rothia | NA | Firmicutes | Veillonella | NA |
| Firmicutes | Streptococcus | NA | Firmicutes | Lachnoanaerobaculum | NA |
| | | | Firmicutes | NA | NA |
| Actinobacteria | Rothia | NA | Firmicutes | Streptococcus | NA |
| Firmicutes | Stomatobaculum | uncultured bacterium | Proteobacteria | Haemophilus | NA |
| | | | Bacteroidetes | Prevotella | Prevotella pallens |
| | | Megasphaera | | | Prevotella sp. oral taxon |
| Firmicutes | Megasphaera | micronuciformis | Bacteroidetes | Prevotella | 299 str. F0039 |
| | | | Firmicutes | Selenomonas 3 | NA |
| | | Megasphaera | Fusobacteria | Fusobacterium | NA |
| Firmicutes | Megasphaera | micronuciformis | Proteobacteria | Haemophilus | NA |
| Firmicutes | Veillonella | NA | Firmicutes | Streptococcus | NA |
| | | | Bacteroidetes | Bergeyella | NA |
| Firmicutes | Veillonella | NA | Proteobacteria | Haemophilus | NA |
| Firmicutes | Selenomonas | NA | Bacteroidetes | Prevotella 7 | NA |
| | | | Firmicutes | Veillonella | NA |
| Firmicutes | Mogibacterium | NA | Firmicutes | Oribacterium | NA |
| Bacteroidetes | Prevotella 7 | NA | Firmicutes | Gemella | NA |
| Bacteroidetes | Prevotella 7 | NA | Firmicutes | Stomatobaculum | uncultured bacterium |

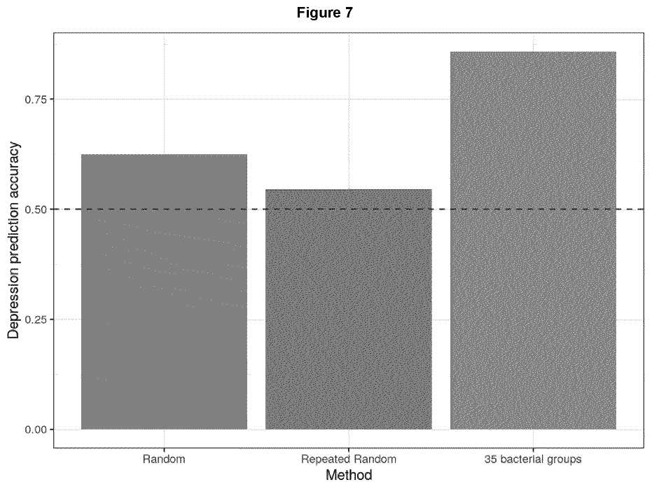

Depression was predicted using a random set of bacterial groups (n=35, see left bar on FIG. 11). Depression was also predicted using 100 random sets of bacterial groups (n=35, see middle bar on FIG. 7). Both approaches performed worse or equivalent to random chance (50% accuracy represented by red dotted line on FIG. 7). This aspect of the invention delivered an unexpected technical benefit (right bar on FIG. 7).

### Example 8

The Structure of the Depressed Microbiome in Individuals with Depression Differs from Control Subjects

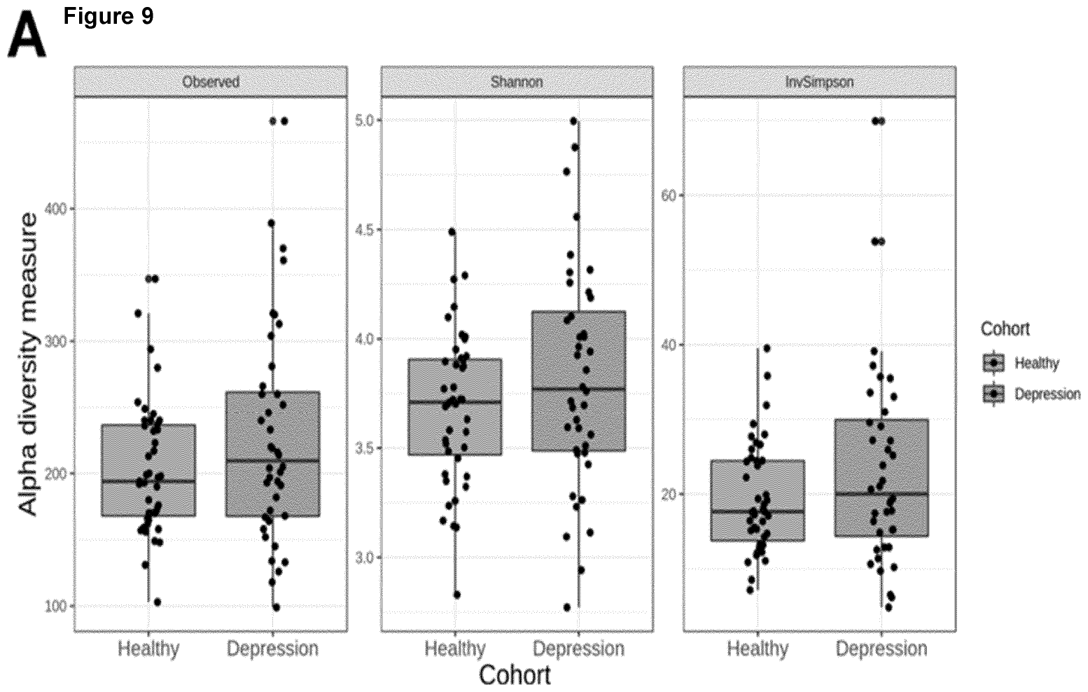

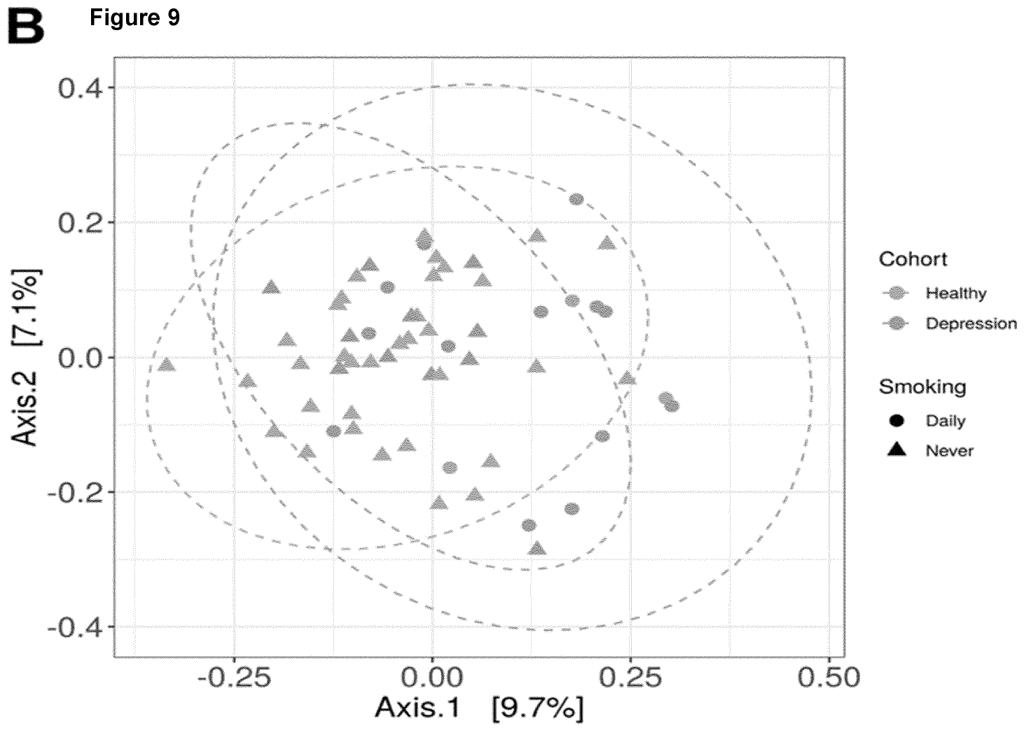

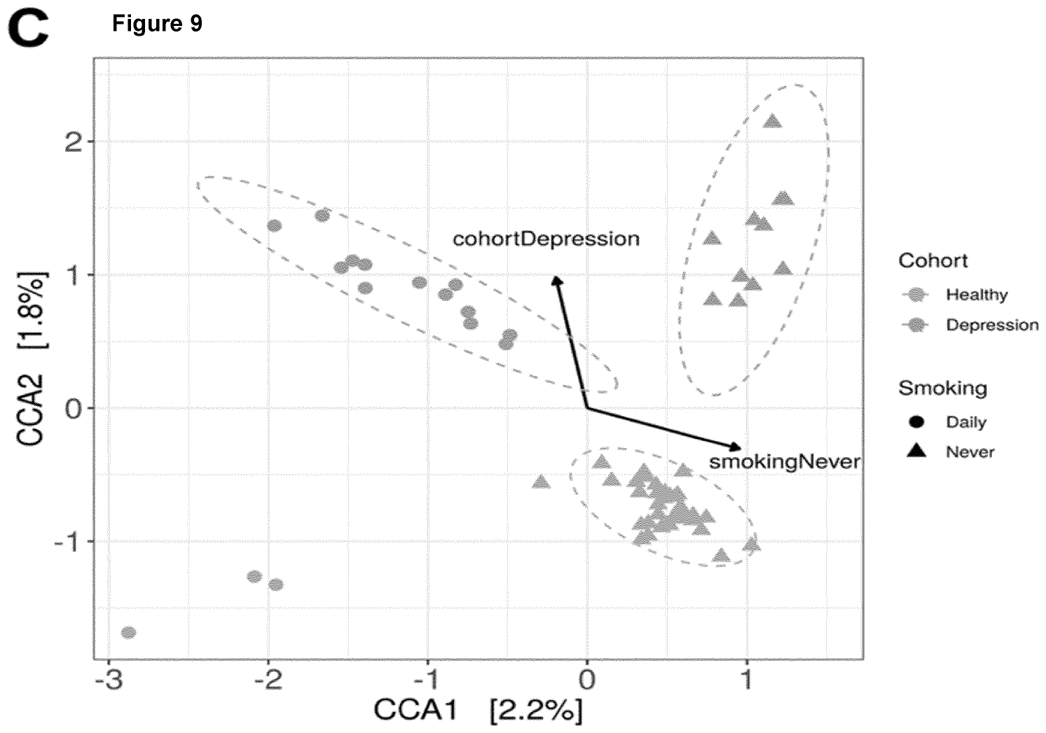

The structure of the salivary microbiome was investigated by estimating the local diversity (α-diversity) of samples (FIG. 9A). To estimate α-diversity, we used the number of observed taxa, and the Inverse Simpson and Shannon diversity indexes. Shannon diversity was significantly more variable in the depressed cohort (F-test; p<0.05), although mean alpha diversity was not significantly different across cohorts for any diversity indices. Smoking is highly prevalent amongst mental health populations compared to the general population so it was not possible to completely match for smoking and, as a consequence, there were significantly more current smokers in the depressed group (p=0016).

CCA was used to test and visualise the effect of depression and smoking on the structure of the oral microbiota (FIG. 9B, 9C). The structure of the oral microbiome differed significantly between the depressed and control cohorts (anova.cca; p=0.002), which clustered into distinct groups. As expected, smoking also significantly altered microbiome composition (anova.cca; p=0.001) (FIG. 2C). The first canonical axis displays a negative correlation with daily smoking (anova.cca; p=0.001), while the second was positively correlated with depression (anova.cca; p=0.002). However, there was no interaction between smoking and cohort status, indicating that the separation of the depressed and control groups is independent of smoking.

FIG. 9 shows the structure of the oral microbiome in subjects with depression is subtly different compared with controls: (A) Shannon diversity is significantly more variable in the depressed cohort (F-test; p<0.05). However, mean alpha diversity was not significantly different across cohorts for any alpha diversity indices (B) Unconstrained analysis of microbiome structure (PCoA) shows no significant clustering between cohorts (C) Constrained analysis of microbiome structure shows significant clustering between cohorts. Canonical Correspondence Analysis (CCA) relates samples (dots) to significant environmental variables (lines). The variation that can be explained by each axis is significant (cca anova; smoking p=0.001; depression p=0.002). The normal data ellipses (also known as concentration ellipses) serve to highlight clusters of samples.

### Example 9

Differential Abundance of Specific Bacterial Taxa in the Salivary Microbiome of Individuals with Depression

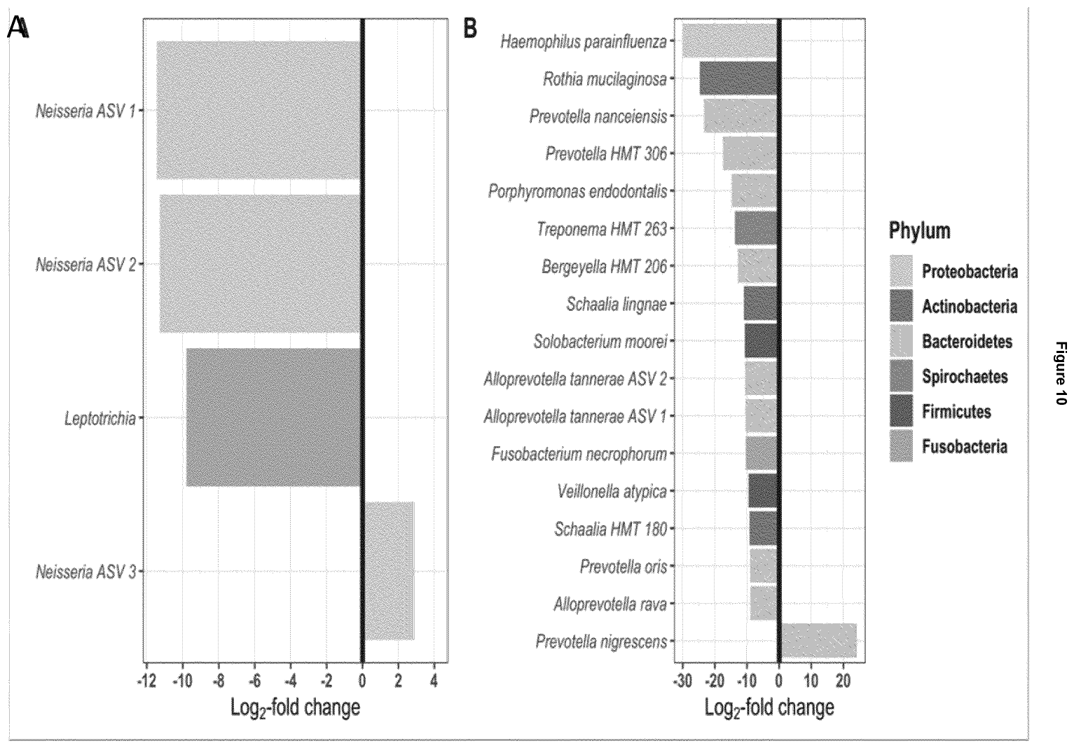

Differential abundance testing of prevalent ASVs found that 21 bacterial taxa were differentially abundant in the depressed cohort relative to the controls. Of these, four ASVs resolved only to the genera level (FIG. 10A), and the remaining 17 were matched to bacterial species (FIG. 10B). From these sequence variants, two were significantly more abundant in depressed subjects, and 19 were significantly less abundant in depressed subjects. Plotting these 21 bacterial taxa shows clear differences in bacterial abundance between control and depressed groups (FIG. 10).

FIG. 10 shows the abundance of amplicon sequence variants is significantly different in the depressed cohort: Differential abundance analysis identified 21 ASVs (that cover 13 genera and 6 phyla) that had a significantly altered abundance in the depressed cohort compared with healthy controls (padj<0.05). A) Differential abundance in 4 ASVs resolving to highest taxonomic level genus; B) Differential abundance in ASVs with species level resolution.

Prevotella nigrescens (Wald test; p<0.001) and Neisseria genera (Wald test; p=0.02) were significantly more abundant in the depressed cohort. ASVs with unique sequences that matched to the same taxonomic group were given arbitrary identifiers to distinguish between them. ASVs in the genera Prevotella, Haemophilus, Rothia, Treponema, Schaalia, Neisseria, Solobacterium, Lepotrichia, Fusobacterium, and Veillonella were less abundant in the depressed cohort (Table 7).

TABLE 7
Statistically significant log2-fold change differences in the abundance
of salivary bacterial taxa between depressed and healthy subjects.

| Genus | Species | log2FoldChange | padj |
|---|---|---|---|
| Haemophilus | parainfluenzae | −30 | <0.001 |
| Rothia | mucilaginosa | −24.71 | <0.001 |
| Prevotella | nanceiensis | −23.37 | <0.001 |
| Prevotella_7 | HMT 306 | −17.54 | <0.001 |
| Porphyromonas | endodontalis | −14.73 | <0.001 |
| Treponema 2 | HMT 263 | −13.78 | <0.001 |
| Bergeyella | HMT 206 | −12.82 | <0.001 |
| Neisseria ASV 1 | | −11.44 | <0.001 |
| Neisseria ASV 2 | | −11.28 | 0.01 |
| Schaalia | lignae | −10.97 | <0.001 |
| Solobacterium | moorei | −10.65 | <0.001 |
| Alloprevotella | tannerae ASV 1 | −10.56 | 0.01 |
| Alloprevotella | tannerae ASV 2 | −10.49 | <0.001 |
| Fusobacterium | necrophorum. | −10.34 | 0.01 |
| Leptotrichia | | −9.81 | 0.01 |
| Veillonella | atypica | −9.47 | 0.05 |
| Schaalia | HMT 180 | −9.23 | <0.001 |
| Prevotella | oris | −9.04 | <0.001 |
| Alloprevotella | rava | −9.02 | 0.01 |
| Neisseria ASV 3 | | 2.89 | 0.02 |
| Prevotella | nigrescens | 24.17 | <0.001 |

### CONCLUSION

This is the first study to clinically investigate the classification potential of the oral microbiome from salivary samples in relation to depression. Composition of the oral microbiome in individuals with depression compared to matched healthy controls was investigated using Next Generation Sequencing and denoised sequent variant bioinformatics analysis. No overall differences in species abundance/richness (alpha-diversity) were found between the two groups, however modest but significant effects of species composition (beta-diversity) were associated with depression in this sample set. Detailed analysis identified 12 differentially abundant ASVs between the two cohorts, two increased and ten decreased in depression, covering three phyla and six genera. Furthermore, inferred metagenomic content analysis with PICRUSt identified a set of pathways that were differentially abundant in the oral microbiome of depressed subjects, and microbial co-occurrence analysis found a set of significant microbial interactions uniquely present in the depressed cohort. Finally, the differences in the oral microbiome are large enough to enable stratification of depressed patients from microbiome census data with the SOM.

Prevotella nigrescens, showing an increased abundance in depression in this cohort, is a bacterial species previously linked with periodontitis. Periodontitis is a bacterial mediated inflammatory disease of gums and teeth associated with a number of systemic, inflammatory conditions including heart disease, diabetes, and rheumatoid arthritis. Evidence is also emerging linking periodontitis with depression. While periodontal disease and depression share a number of environmental risk factors such as age, low socioeconomic status, smoking and alcohol consumption, sleep deprivation and stress, predisposition to periodontitis and depression also share common genetic polymorphisms. The BDNF GG genotype has been shown to correlate with the levels of BDNF protein and the chemokine CXCL10, associated with chronic periodontitis, and 5HTT promotor polymorphism, 5HTTLPR, associated with stress reactivity, was analysed in cases of aggressive periodontitis the SS genotype and S allele was significantly associated with aggressive periodontitis, the SS genotype was also significant in an elderly group with periodontal disease. Inflammation plays key role in both periodontitis and depression and antidepressants have been shown to reduce the inflammatory effect of periodontitis and disease severity.

Surprisingly, the majority of the differentially abundant species were decreased in the depression cohort, suggesting a decrease in the opportunistic pathogens identified. Further downstream analyses are required to determine the consequences of these alterations in the microbiome with respect to inflammation, however the microbial community is complex and imbalances in microbiota composition, or loss of diversity may lead to systemic and neuropathological consequences.

Many previous animal models support the role of the gut microbiome in the pathology of psychiatric illness including depression, and emerging studies are demonstrating the therapeutic effects of probiotics. The positive probiotic effects of specific bacteria were investigated in a rat MS model of depression. Before treatment, the rat MS model showed a decreased motivational state in the forced swim test, decreased levels of noradrenaline in the brain, increased peripheral IL-6 levels and upregulated corticotrophin-releasing factor (CRF) mRNA levels. Following treatment with probiotic Bifobacterium infantis, depressive-like behaviours were reversed, and IL-6, CRF and NA levels restored.

A very limited number of human studies have investigated the role of the gut microbiome specifically in depression. Active depression was associated with decreased alpha diversity, increased abundance of Enterobacteriaceae and Alistipes, and a reduced level of Faecalibacterium in the gut microbiome compared to healthy controls. Naseribafrouei et al. (2014) reported significantly increased Bacteroidales, while Lachnospiraceae were significantly decreased in the gut microbiome isolated from faecal samples from depressed patients compared to healthy individuals. Another clinical study demonstrated that oral administration of Bifidobacterium longum and Lactobacillus helveticus combination, taken over a 30 day period, improved Hospital Anxiety and Depression Scale scores.

The present invention has not identified the same differently abundant bacteria. Chronic diseases have a specific microbiome signature, predominantly determined from the gut and oral microbiome, the main sites of microbiota for novel biomarker discovery for disease diagnosis and treatment. The diversity and composition of the oral microbiome is a close representation of the upper gastrointestinal (GI) tract. Comparison of the microbial profile of stimulated saliva, gastric fluid and faecal samples indicates that species richness is comparable between all three sample types, however both saliva and gastric fluid differed in community structure from the faecal sample. Interestingly, the individual variation of the microbiome was greater in the faecal sample compared to the other two samples, indicating that saliva may be a more stable source of microbial material for biomarker discovery.

Microbiota produce specific nucleic acids and metabolites with systemic effects including gene activation through epigenetic mechanisms and this interaction has changed over evolution. The cause-consequence relationship between disease and the composition of the microbiome is still not fully understood, but a number of studies have investigated the role of host genetics through twin studies. To investigate how host genome and environmental factors influence the microbiome, faecal samples were collected from female twins and the microbial community analysed; results indicated shared microbial communities between families, but environmental factors such as obesity are associated with changes at phylum level. Future analysis of this cohort could incorporate host genetics to determine the level of interaction between genetics and the composition of the microbiome.

Analysis focused on the most severe cases of depression compared to controls, reporting never or very rarely experiencing any symptoms of depression representing the extremes on either side of the scale which may explain why clear differences were observed between the groups. This study has been designed to collect samples from a large number of individuals at one time point. While repeated samples may offer a more comprehensive analysis of diversity, there is evidence from gut microbiome studies that individual microbiomes remain relatively stable overtime. Less information is available on the stability of the oral microbiome, however a recent study concluded no diurnal variation within individual salivary microbiome samples over 24 hours.

The oral microbiome is one of the most diverse microbiomes in the human body, and has a significant bearing on the microbiota found in the rest of the gastrointestinal tract, potentially playing a key role in health and disease. Oral dysbiosis has been linked not only to oral disease, but to other systemic diseases with an underlying inflammatory aetiology, including inflammatory bowel disease.

There is strong evidence from both preclinical animal models and clinical studies that depression is associated with the composition of the gut microbiome, including altered diversity and differential abundance of certain bacterial taxa. Our data now adds to these previous results and suggests that depression also confers apparent and detectable changes in the salivary microbiome. Of the 21 bacterial taxa that were differentially abundant in the oral cavity, the majority (n=19) were decreased in individuals with depression compared to controls, similar to previous reports with the gut microbiome. While microbiome composition is site-specific, there is evidence to indicate a degree of overlap and crosstalk between the oral and gut microbiomes. Oral microbiota are enriched within different gastrointestinal locations in individuals with treatment-naive microbiome in new-onset Crohn's disease (CD), suggesting that oral bacteria may colonise the gut and contribute to chronic inflammatory disease. It is also very probable that microbes and their metabolites in the oral cavity may translocate or leak thorough a compromised blood-brain barrier, leading to neuroinflammation, an important feature in the aetiology of depression.

The mouth is highly vascularized and bacteraemia due to bacterial translocation across the epithelial mucosa is an everyday event. This ‘mobile microbiome’ has the potential to cause metastatic infection, injury and inflammation. The recent and exciting demonstration of a ‘dormant blood microbiome’ that is disturbed in patients with cardiovascular disease (CVD) versus healthy controls further highlights the importance of haematogenous spread of bacteria and the development of disease at distal sites.

Additionally, charting the oral microbiome in Rheumatoid Arthritis (RA) patients has revealed markers associated with both risk and therapeutic response. Microbiome profiling was carried out on samples collected from patients with RA and healthy controls, and RA patients showed significant detectable changes in both their gut and oral microbiome. The oral microbiome has also been characterised for patients with pancreatic cancer. Specific bacterial taxa, including Porphyromanas gingi were associated with increased pancreatic cancer risk, while the phyla Fusobacteria was associated with deceased risk of pancreatic cancer.

Signs of oral dysbiosis were evident in our depressed cohort with ASVs corresponding to Prevotella nigrescens, a bacterial species previously linked to periodontitis and Th17 immune responses in-vivo, demonstrating the most increased abundance in depression. While periodontal disease and depression share a number of environmental risk factors such as age, low socioeconomic status, smoking and alcohol consumption, sleep deprivation and stress, predisposition to chronic and aggressive periodontitis also shares common genetic polymorphisms with depression in relation to the genes for BDNF, CXCL10 and 5HTT57-59. Inflammation plays a key role in both periodontitis and depression, and the presence and abundance of specific microbiota within the oral cavity that could contribute to both periododontitis and depression through a common host inflammatory response is highly probable. On this basis, the well described anti-inflammatory effects of antidepressants may help explain, at least in part, their efficacious effects in this context.

Strikingly, we observed a widespread reduction in several oral taxa in the depressed cohort versus controls, including known commensals. The largest difference was found with Heamophilus parainfluanzae, a common species found throughout the oral cavity which has anti-proliferative effects against cancereous cells but can also behave as an opportunistic pathogen. Rothia mucilaginosa is also a common commensal in the oral cavity and produces enterobactin that reduces the growth of certain strains of cariogenic Streptococcus mutans and pathogenic strains of Staphylococcus aureus. Reductions in this species, as observed in the depressed cohort, are associated with oral dysbiosis. Furthermore, Schaalia lingnae, formerly known as Actinomyces lingnae, has been identified as one of the core microbiota associated with a healthy oral cavity and is was found to be decreased in individuals with depression.

We can speculate that the lower abundance observed amongst organisms considered part of the normal microbiota may predispose to a more pathogenic or inflammatory microbial composition within the oral site of depressed subjects. In particular, lower levels of actinomyces have previously been linked with high anxiety and cortisol levels in adolescents and may be a marker of hyperactivation of the hypothalamic-pituitary-adrenal (HPA) axis, also common in the pathophysiology of depression. A number of taxa in the genus Prevotella have also been negatively associated with depression and psychological distress and Haemophilus and Neisseria taxa are also depleted in the oral microbome of individuals with rheumatoid arthritis, possibly indicative of an inflammatory state. It is, however, important to note that we did also observe a higher abundance of some species in the healthy cohort, including Solobacter moorei, Alloprevotella tannerae and Porphyroomonas endodontalis that have been previously described in the context of halitotsis and periodontal disease.

Yet, despite such observations, our understanding of the specific role of these and indeed other oral microbes in human health and disease remains poor, and it is unclear of the extent to which specific lineages with a heightened capacity to cause disease exist alongside strains of the same species that are more positively associated with health; this has been observed with other human commensal bacteria. Such intraspecies differences can complicate interpretation of microbiome changes in health and disease, alongside complex interaction networks between taxa in disease states that we do not currently understand.

Smoking impacts directly and indirectly on oral bacteria. In this sample set, a high portion of individuals with severe depression reported daily or occasional smoking, in comparison to a very low prevalence of smoking in healthy individuals. During sample selection, priority was placed on depression, with smoking status matched where possible. In our cohort, both depression and smoking significantly altered the microbial community composition in saliva as would be expected. The effects observed, however, did not appear to overlap and altered the microbiome in different ways based on the separation observed following CCA analysis. Furthermore, differential abundance in individual taxa were identified after controlling for smoking status. As a result, the effects of depression observed here were independent and not an artefact of smoking status.

Oral health and hygiene habits also impact on the oral microbiome. In this study, we did not collect data on oral health and have not controlled for this variable in the current report. There is a documented association between depression and poor oral health but the relationship is complex and while depression may lead to poor oral health in some cases, in others a lack of personal care may precede depression. It is possible that the differences in the oral microbiome that we observed are not directly attributable to depression in all cases but a secondary consequence of poor oral health. It is also plausible that poor oral hygiene may be a precursor to poor overall health and systemic inflammation, a risk factor for depression. This link has received considerable attention in the context of cardiovascular disease but not been extensively studied to date for mental health. As noted above, bacterial species linked to periodontal disease have been found at higher levels in both healthy (Solobacter moorei and Alloprevotella tannerae) and depressed individuals (Prevotella nigrescens) so the relationship between oral health, depression and changes in the oral microbiome is complex and will require much further investigation.

Host behaviours including dietary factors such as sugar intake can also alter the oral microbiome. Eating behaviour associated with depression can include consuming less food eating more or no change, and the composition of an individual's diet will be highly variable⁵⁴. Furthermore, the diet of the control cohort will naturally vary too, but as no information on food intake was recorded in the present we cannot determine the possible impact of diet on the differences observed.

Saliva is a cost effective non-invasive biomarker source that offers collection, handling and economic advantages over blood or faecal samples. Saliva is a heterogeneous fluid made up of water, proteins and small inorganic substances and essential for digestion, lubrication and acts a barrier to pathogens. Three key salivary glands are the source of nearly 90% of saliva fluid and these glands are surrounded by capillaries, and are highly permeable, with the potential to absorb blood based biomarkers of disease both local, systemic or infectious, suggesting saliva fluid may contain vital disease information.

1-29. (canceled)

30. A method of treating depression in a subject, the method comprising the steps of:
(a) providing a sample of the microbiome from the subject;
(b) determining the quantitative level or presence of one or more bacterial species in the sample; and
(c) diagnosing depression based on the quantitative level or presence of the one or more bacterial species in the sample;
wherein the one or more bacterial species is selected from one or more of the phyla:
(i) Firmicutes;
(ii) Proteobacteria;
(iii) Bacteroidetes;
(iv) Fusobacteria;
(v) Actinobacteria; and
(vi) Spirochaetes;
wherein the quantitative level or presence of the one or more bacterial species in the sample is indicative of depression in the subject; and
(d) after depression is diagnosed in the subject, treating the subject for depression.

31. The method according to claim 30, wherein one or more of the one or more bacterial species is selected from the phylum Firmicutes and is further selected from one or more of the genera:
(i) Solobacterium;
(ii) Veillonella;
(iii) Streptococcus;
(iv) Lachnoanaerobaculum;
(v) Selenomonas_3;
(vi) Oribacterium;
(vii) Gemella;
(viii) Stomatobaculum;
(ix) Megasphaera;
(x) Selenomonas;
(xi) Carnobacteria;
(xii) Erysipelotrichia;
(xiii) Mogibacterium; and
(xiv) Granulicatella.

32. The method according to claim 30, wherein one or more of the one or more bacterial species is selected from the phylum Proteobacteria and is further selected from one or more of the genera:
(i) Haemophilus;
(ii) Pasteurella;
(iii) Neisseria;
(iv) Aggregatibacter;
(v) Neisseria ASV 1;
(vi) Neisseria ASV 2; and
(vii) Neisseria ASV 3.

33. The method according to claim 30, wherein one or more of the one or more bacterial species is selected from the phylum Bacteroidetes and is further selected from one or more of the genera:
(i) Prevotella;
(ii) Prevotella_7;
(iii) Bergeyella;
(iv) Porphyromonas;
(v) Flavobacteria;
(vi) Alloprevotella; and
(vii) Prevotella_6.

34. The method according to claim 30, wherein one or more of the one or more bacterial species is selected from the phylum Bacteroidetes and is further selected from the genus Fusobacterium.

35. The method according to claim 30, wherein one or more of the one or more bacterial species is selected from the phylum Fusobacteria and is further selected from one or more of the genera:
(i) Leptotrichia; and
(ii) Fusobacterium.

36. The method according to claim 30, wherein one or more of the one or more bacterial species is selected from the phylum Actinobacteria and is further selected from one or more of the genera:
(i) Rothia;
(ii) Actinomyces; and
(iii) Schaalia.

37. The method according to claim 30, wherein one or more of the one or more bacterial species is selected from the phylum Spirochaetes and is further selected from the genus Treponema_2.

38. The method according to claim 31, wherein one or more of the one or more bacterial species is selected from one or more of the species:
(i) Veillonella dispar;
(ii) Veillonlla parvula;
(iii) Veillonella rogosae;
(iv) Veillonella atypica;
(v) Streptococcus australis;
(vi) Streptococcus infantis;
(vii) Streptococcus sanguinis;
(viii) Streptococcus parasanguinis;
(ix) Streptococcus oralis;
(x) Gemella haemolysans;
(xi) Gemella sanguinis.
(xii) Megasphaera micronuciformis; and
(xiii) Selenomonas sputigena.

39. The method according to claim 32, wherein one or more of the one or more bacterial species is selected from the species:
(i) Haemophilus parainfluenzae;
(ii) Neisseria mucosa;
(iii) Neisseria pharynges;
(iv) Neisseria perflava; and
(v) Neisseria subflava.

40. The method according to claim 33, wherein one or more of the one or more bacterial species is selected from the species:
(i) Alloprevotella tannerae;
(ii) Prevotella nigrescens;
(iii) Prevotella oris;
(iv) Prevotella pallens;
(v) Prevotella sp. oral taxon 299 str. F0039;
(vi) Prevotella nanceinsis;
(vii) Prevotella_6 salivae;
(viii) Porphyromonas endodontalis;
(ix) Bergeyella HMT 206;
(x) Prevotella_7 sp. oral clone GI059;
(xi) Prevotella_7 jejuni;
(xii) Prevotella_7 melaninogenica; and
(xiii) Prevotella_7 HMT 306.

41. The method according to claim 35, wherein one or more of the one or more bacterial species is selected from the species Fusobacterium necrophorum ssp. Necrophorum.

42. The method according to claim 36, wherein one or more of the one or more bacterial species is selected from the species:
(i) Schaalia lignae
(ii) Schaalia HMT 180; and
(iii) Rothia mucilaginos.

43. The method according to claim 37, wherein one or more of the one or more bacterial species is selected from the species Treponema sp. 5:22:BH022.

44. The method according to claim 30, wherein the sample of the microbiome is a sample of the oral microbiome.

45. The method according to claim 30, wherein one or more of the one or more bacterial species is selected from one or more of the genera:
(i) Solobacterium;
(ii) Veillonella;
(iii) Streptococcus;
(iv) Lachnoanaerobaculum;
(v) Selenomonas_3;
(vi) Oribacterium;
(vii) Gemella;
(viii) Stomatobaculum;
(ix) Megasphaera;
(x) Selenomonas;
(xi) Carnobacteria;
(xii) Erysipelotrichia;
(xiii) Mogibacterium;
(xiv) Granulicatella.
(xv) Haemophilus;
(xvi) Pasteurella;
(xvii) Neisseria;
(xviii) Aggregatibacter;
(xix) Neisseria ASV 1;
(xx) Neisseria ASV 2;
(xxi) Neisseria ASV 3;
(xxii) Prevotella;
(xxiii) Prevotella_7;
(xxiv) Bergeyella;
(xxv) Porphyromonas;
(xxvi) Flavobacteria;
(xxvii) Alloprevotella;
(xxviii) Prevotella_6;
(xxix) Fusobacterium;
(xxx) Leptotrichia;
(xxxi) Rothia;
(xxxii) Actinomyces;
(xxxiii) Schaalia; and
(xxxiv) Treponema_2

46. The method of claim 30, wherein one or more of the one or more bacterial species is selected from one or more of the species:
(i) Veillonella dispar;
(ii) Veillonlla parvula;
(iii) Veillonella rogosae;
(iv) Veillonella atypica.
(v) Streptococcus australis;
(vi) Streptococcus infantis;
(vii) Streptococcus sanguinis;
(viii) Streptococcus parasanguinis;
(ix) Streptococcus oralis.
(x) Gemella haemolysans;
(xi) Gemella sanguinis;
(xii) Megasphaera micronuciformis;
(xiii) Selenomonas sputigena;
(xiv) Haemophilus parainfluenzae;
(xv) Neisseria mucosa;
(xvi) Neisseria pharynges;
(xvii) Neisseria perflava;
(xviii) Neisseria subflava;
(xix) Alloprevotella tannerae;
(xx) Prevotella nigrescens;
(xxi) Prevotella oris;
(xxii) Prevotella pallens;
(xxiii) Prevotella sp. oral taxon 299 str. F0039;
(xxiv) Prevotella nanceinsis;
(xxv) Prevotella_6 salivae;
(xxvi) Porphyromonas endodontalis;
(xxvii) Bergeyella HMT 206;
(xxviii) Prevotella_7 sp. oral clone GI059;
(xxix) Prevotella 7jejuni;
(xxx) Prevotella_7 melaninogenica;
(xxxi) Prevotella_7 HMT 306;
(xxxii) Fusobacterium necrophorum ssp. Necrophorum;
(xxxiii) Schaalia lignae;
(xxxiv) Schaalia HMT 180;
(xxxv) Rothia mucilaginosa; and
(xxxvi) Treponema sp. 5:22:BH022.

47. A method of diagnosing depression in a subject, the method comprising the steps of:
(a) providing a sample of the microbiome from the subject;
(b) determining the quantitative level or presence of one or more bacterial species in the sample; and
(c) diagnosing depression based on the quantitative level or presence of the one or more bacterial species in the sample;
wherein the one or more bacterial species is selected from one or more of the phyla:
(i) Firmicutes;
(ii) Proteobacteria;
(iii) Bacteroidetes;
(iv) Fusobacteria;
(v) Actinobacteria; and
(vi) Spirochaetes;
wherein the quantitative level or presence of the one or more bacterial species in the sample is indicative of depression in the subject.
